(19) United States
(12) Patent Application Publication
(10) Pub. No.: US 2010/0015203 A1
(43) Pub. Date: Jan. 21, 2010

(54) BIOCOMPATIBLE FIBRINOGEN MATRIX ON A SOLID SUPPORT

(75) Inventors: Pentti Tengvall, Linkoping (SE); Ella Cathrine Vikinge, Brokind (SE)

Correspondence Address: MORRISON & FOERSTER LLP, 425 MARKET STREET, SAN FRANCISCO, CA, 94105-2482, US

(73) Assignee: ADDBIO AB, Linkoping (SE)

(21) Appl. No.: 12/528,799

(22) PCT Filed: Feb. 26, 2008
(86) PCT No.: PCT/SE2008/050213

§ 371 Date: Aug. 26, 2009

Related U.S. Application Data
(60) Provisional application No. 60/903,518, filed on Feb. 27, 2007.

(51) Int. Cl. A61F 2/00; A61K 31/66; A61K 31/675

(52) U.S. Cl. 424/423; 514/108; 514/94

(57) ABSTRACT

The present invention provides a non-clottable matrix on a solid support comprising immobilized and crosslinked fibrinogen. The matrix may further comprise, in and/or on the matrix, one or several biologically active compound(s) and/or pharmacological substance(s). The matrix may be composed of one or several fibrinogen layer(s). The solid support according to the present invention may be selected from the group consisting of orthopaedic devices, implants, stitches, stents, pins, screws, plates, and sutures.

The present invention relates to a biocompatible fibrinogen matrix on a solid support. The invention also relates to said biocompatible fibrinogen matrix on a solid support further comprising in and/or on the matrix one or several biologically active compound(s) and/or pharmacological substance(s). The solid support is e.g. an implant device or a suture thread, and the biologically active compound(s) and/or pharmacological substances are intended to be exposed, and/or delivered from the matrix, to the surrounding tissues in a mammalian body.

### BACKGROUND OF THE INVENTION

Implants, in particular orthopaedic implants, coated with proteins, such as fibrinogen, and bisphosphonates have been disclosed in the U.S. Pat. No. 7,163,690 as well as in EP 04001331. Suture threads coated with crosslinked fibrinogen and pharmacological substances that inhibit tissue break-down have been disclosed in the International Patent Application WO 06/126926.

Fibrinogen is a flexible protein having 3 structurally bound calcium ions, and it can easily be used for building a matrix by crosslinking layers of fibrinogen. Further, it has a low immunoactivation. In the prior art fibrinogen matrices, the matrix is constructed by immobilizing a layer of protein/fibrinogen on top of a previously immobilized layer, and repetition of this process until the desired thickness and amount of protein/fibrinogen is achieved. Fibrinogen is a blood protein with its primary function in the blood clotting cascade. When the suggested methods are used for the coupling of the fibrinogen proteins to each other, the sites on the protein involved in the clotting process may be used. This in itself may render the protein less likely to partake in blood clotting, when being part of the matrix. Also the fact that the protein is restricted in its mobility by the coupling to surrounding proteins inflicts on its abilities to partake in this process. As the matrix is extremely small and the state of the individual fibrinogen/proteins is not easily investigated, it cannot be ruled out that fibrinogen of the prior art matrices can in some way take part in steps of the coagulation cascade.

However, in some applications of fibrinogen matrices it may be desirable to ensure that there is no risk of the fibrinogen components, building up the matrix, contributing to the building of blood clots, when a clot implies a network able to stop flowing blood or liquid. For instance, this may be required by regulatory authorities.

### DESCRIPTION OF THE INVENTION

The present invention provides a cross-linked fibrinogen matrix immobilized on a solid support, such as an implant device or a suture thread, wherein it has been ensured that the fibrinogen of the matrix is non-clottable. The invention further provides such a matrix comprising in and/or on the matrix one or several biologically active compound(s) and/or pharmacological substance(s), such as bisphosphonates, proteins, peptides, steroids, hormones, bone morphogenic proteins, matrix metallo-proteinase inhibitors etc, which are intended to be exposed to and/or delivered to the surrounding tissues upon insertion of the matrix into a mammalian body.

Thus, the present invention is directed to a matrix on a solid support comprising immobilized and crosslinked non-clottable fibrinogen.

The invention is further directed to such a matrix further comprising in and/or on the matrix one or several biologically active compound(s) and/or pharmacological substance(s).

In an embodiment of the invention the non-clottable fibrinogen matrix is composed of one or several fibrinogen layer(s), such as those wherein the several fibrinogen layers are selected from 2 to 100 layers. Examples of the number of fibrinogen layers comprise 2, 3, 4, 5, 6, 7, 8, 9, 10, 11, 12, 13, 14, 15, 16, 17, 18, 19 or 20 layers, as well as any number of layers between 20-50 or 50-100.

In another embodiment of the invention, the fibrinogen has been rendered non-clottable stepwise during the production of the matrix.

In yet another embodiment of the invention, the fibrinogen has been rendered non-clottable prior to the production of the matrix.

In still another embodiment of the invention, the fibrinogen of at least the outer layer(s) has been rendered non-clottable after the matrix has been constructed.

In a further embodiment of the invention, the fibrinogen of the matrix is selected from blood-derived fibrinogen and recombinant fibrinogen.

The solid support carrying the fibrinogen matrix of the invention is e.g. selected from the group consisting of orthopaedic devices, implants, stitches, pins, screws, plates, stents and sutures.

In an embodiment of the invention, the fibrinogen matrix is attached to the solid support via a non-fibrinogen protein or substance, i.e. a non-fibrinogen protein, such as serum albumin, or so called adhesive proteins, such as mussel adhesive proteins, or mimetics thereof, or adhesive carbohydrates. The non-fibrinogen protein or substance is attached to the surface of the solid support, and fibrinogen is attached to the non-fibrinogen protein or substance.

The one or several pharmacological substance(s) comprised in and/or on the fibrinogen matrix of the invention is (are) e.g. selected from the group consisting of tetracyclines, chemically modified tetracyclines, synthetic matrix metalloproteinase inhibitors, including those of the hydroxamate subgroup; cyclooxygenase inhibitors, including cyclooxygenase 2 specific inhibitors; nuclear factor kappa B inhibitors; lipooxygenase inhibitors; corticosteroids including glucocorticoids; macrolide antibiotics; hydroxymethylglutaryl coenzyme A reductase inhibitors (statins); angiotensin converting enzyme (ACE) inhibitors; angiotensin 11 receptor blockers (ARBs); bone morphogenic proteins (BMPs); aprotinin; gabexate mesilate; sulfasalazine; inhibitors of tumour necrosis factor alpha; and transforming growth factor beta inhibitors, and bisphos phonates including compounds with the generic formula

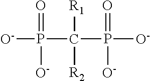

wherein R₁ and R₂ are independently selected from the group consisting of —H, —OH, —Cl, —CH₃,

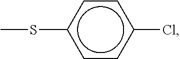

### —CH₂—CH₂—NH₂, —(CH₂)₅—NH₂, —(CH₂)₂N(CH₃)₂,

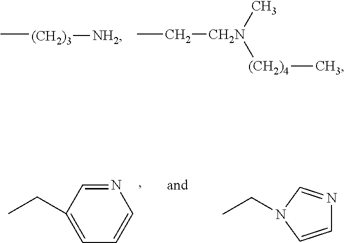

as well as pharmaceutically acceptable salts and hydrates thereof.

Specific examples of bisphosphonate compounds of the above formula are:

| Agent | R₁ side chain | R₂ side chain |
|---|---|---|
| Etidronate | —OH | —CH₃ |
| Clodronate | —Cl | —Cl |
| Tiludronate | —H | 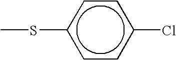 |
| Pamidronate | —OH | —CH₂—CH₂—NH₂ |
| Neridronate | —OH | —(CH₂)₅—NH₂ |
| Olpadronate | —OH | —(CH₂)₂N(CH₃)₂ |
| Alendronate | —OH | —(CH₂)₃—NH₂ |
| Ibandronate | —OH | 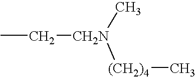 |
| Risedronate | —OH | 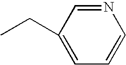 |
| Zoledronate | —OH | 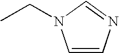 |

### Rendering the Fibrinogen Matrix Non-Clottable

Rendering the fibrinogen matrix biocompatible (that is, non-clottable) can be achieved by modifications to all or less than all (such as the outer layer(s)) the individual fibrinogen proteins constituting the matrix. Such modifications of fibrinogen may be of chemical nature, sterical changes, or any other changes or combinations thereof giving the desired effect, e.g. rendering the matrix and its constituents unable to form a clot. Three principle ways are outlined; either by 1) use of fibrinogen that is rendered inactive prior to the matrix construction, or by 2) deactivation of fibrinogen during matrix construction (in between the other steps, or as one late step), or 3) after matrix construction has been completed.

In case of way 1), this may have been done by the provider of the fibrinogen, or by the manufacturer of the matrix. Fibrinogen may be treated in the dry phase, for example by heat, UV-irradiation, radioactivity or other suitable means. The principle behind this is to use a method that adds energy to the protein, or to water/crystal water on/at the protein/in the protein surrounding. Absorbed energy may lead to changes in binding patterns between the atoms in the protein, and thereby change chemical, sterical, etc patterns required for recognition and fulfillment of natural purpose. Also comprised is the use of fibrinogen rendered non-clottable simply by the procedure by which it is purified from blood, or, if recombinant protein is used, from the used liquid. It is well-known that purification of blood proteins requires that great care is taken to preserve the protein properties, such as clottability. Thus, modifying purification procedures may produce fibrinogen suitable for the present use. Also, another way obvious to those skilled in the art is to introduce mutations in the genome to change the fibrinogen expressed to a non-clottable one. The use of such protein is comprised by the invention, and the methods to be used are known to those skilled in the art.

In case of way 2), the modification of the fibrinogen during construction of the matrix can be done in a step-wise manner following fibrinogen or drug/substance immobilisation-steps, or after the last substance (fibrinogen or drug/substance) to be immobilised by chemical means. In both cases, substances rendering activated carboxyl and/or amine groups inactive can be used. Such substances include those reacting spontaneously with activated carboxyl groups, such as internal fibrinogen amine groups, ethanol amine (NH₂CH₂CH₂OH) or amino acids, peptides, proteins, thiols, etc. The supplied inactivating substance is preferably small and will be able to penetrate through the matrix and inactivate activated groups within the entire matrix. Optionally, even amine groups at the fibrinogen, such as the amine terminals including the thrombin binding sites, can be rendered non-clottable by chemical means. Chemical substances suitable for this typically include aldehydes, periodate, amine reactive NHS esters, etc.

In case of way 3), all or only the uppermost layer(s) of fibrinogen is rendered non-clottable. This can be accomplished by blocking the thrombin binding sites on fibrinogen by amine binding substances, such as those mentioned above, or by highly specific affinity ligands. A rational behind deactivating only the uppermost layer may be that even if thrombin binding sites in lower layers of the fibrinogen matrix could be activated, thrombin will not be able to penetrate into the matrix due to its size.

Thus, a dense matrix may only require deactivation of uppermost layer(s), whereas a less dense matrix, i.e. one in which spacers are used between fibrinogen layers, may require that fibrinogen through the entire matrix be rendered non-clottable.

Alternatively, or to further diminish the risk of having fibrinogen biofunctionality left in the fibrinogen matrix, the matrix is, after preparation, treated so as to change the inter and intra fibrinogen chemical binding patterns. For example this may be achieved by use of gamma-irradiation. The radiation energy is taken up by amino acids and/or residual water left in the matrix, such as crystal water at and/or close to the fibrinogen. Other possible means are alpha or beta-radiation, etc. Energy absorbed changes electron configuration, and may be transferred and thus change chemical binding patterns in and between fibrinogen, changing the appearance and thus functionality of the fibrinogen constituents of the matrix. The radiation dose required varies with amount of water residues, typically 25 kGy will be appropriate when drying has been performed as described. With more extensive drying, dosage will possibly need to be increased.

Incorporation of Biologically Active and/or Pharmacological Substance(s)

The biologically active and/or pharmacological substance(s) may be incorporated into the matrix in one or several steps in between or concomitant with the protein cross-linking (matrix construction), or in one or several step(s) after matrix is completed. The biologically active and/or pharmacological substance or drug is incorporated into the matrix in the wet phase, preferably in a pH-adjusted suitable buffer. Mechanisms retaining the drug/substance within the matrix are covalent interactions, electrostatic interactions, hydrophobic, van der Waals, etc, or combinations thereof. Incorporation is thus achieved by use of activating substances such as carbodiimides, e.g. EDC, or incorporation of chemical spacers and/or linkers such as aldehydes, maleimide, sulfo-SMCC, reductive amination chemistry, photoactivable immobilization chemistry or Mannich condensation chemistry, or by use of buffers with controlled pH, salt, surfactant etc concentrations, making the incorporation of the drug happen. The drug/substance is retained in the matrix when this is rinsed at the end of the incorporation procedure.

### DETAILED DESCRIPTION OF EMBODIMENTS AND PRODUCTION METHODS

The protein fibrinogen forming the immobilized fibrinogen matrix according to the invention is selected for its non-foreign and structural properties. It is not desired that the protein keeps its, for the purpose of the present invention, unappropriate properties As the protein of the matrix is fibrinogen, it is desirable to make sure that the protein cannot clot when inserted into the body environment. Fibrinogen engagement in the blood coagulation cascade is by thrombin cleaving off Fibrinopeptide A, and subsequent potentially cleavage off of Fibrinopeptide B, from central amino-terminals, rendering the fibrin monomer accessible for binding to D-globules of other fibrin monomers. The weak network formed is strengthened by activated Factor XIII, and the fibrin network resulting is important for blood clotting. Depending on where in the body the coated device is placed, a risk is introduced of unintentional coagulation. To avoid this risk, the sites on the fibrinogen involved in fibrin monomer polymerisation are modified. The carboxyl terminals may be inactivated by incubation with amino acids, peptides, proteins, thiols, or ethanolamine. For example, ethanolamine (NH₂CH₂CH₂OH), 1 mg/ml is incubated for 30 minutes with the matrix after the last step of covalent coupling (fibrinogen or a covalently coupled drug). Optionally, even amine groups may be inactivated. This is achieved by use of aldehydes, periodate, amine reactive NHS esters, such as H₂N—R—X wherein —X is selected from the group consisting of —NH₂, —OH, and —CO₂CH₂CH₃; and R is selected from the group consisting of an alkyl group and an alkyl ether group; wherein, when —X is —NH2 or —CO₂CH₂CH₃, R comprises from 1 to 20 carbon atoms; and when —X is —OH, R comprises from 4 to 20 carbon atoms, etc. Typically, 1 mg/ml of NHS ester is mixed with the matrix for 30 minutes to achieve inactivation. Successful amine group inactivation is qualitatively evaluated by use of biotin-labelled NHS ester dye (Amersham). Either or both amine and carboxylgroup inactivation may be performed. It may also be possible to render fibrinogen non-clottable by reduction of one or several internal disulfide bridges, such as gamma 326Cys-gamma 339Cys intrachain disulfide bond.

A coated implant device, preferably but not necessarily, has a rough surface and several types of coating layers including a suitable protein matrix, and one or more biologically active component(s) or pharmacological substance(s) or drugs embedded therein. One important feature enabled by the present invention is that the drug substances can be associated with the matrix protein by covalent binding, electroststic interactions hydrophobic ditto, van der Waals forces, or combinations thereof. Thus, specific chemical groups available for e.g. covalent coupling are not a requirement. By modifications of ion concentration and pH, the substances can be brought to associate with the fibrinogen matrix and retained.

The implant device may be any suitable material such as stainless steel, titanium, Ti-alloys, CoCr-alloys, Zr and ZrO₂, Nb and NbO₂, Ta and TaO₂ and their alloys, Sr and Hf and their alloys, biopolymers such as polyurethanes, polycarbonates, polysiloxanes, polydimethylmethacrylate, polysulphones, polylactic acid/polyglycolic acid blends, hydroxybutyrates, polycaprolactones, dextrans, polyvinylalcohols, hydrogels, apatites such as hydroxyapatite or tri-calcium phosphate, plaster of paris, metal oxides, or any other material that provides sufficient support. The implant may be any device, for example a coated or uncoated metallic screw, chamber, plate, pin, rod, cylinder, net, or a polymeric pad. As an illustrative example, the implant device is a screw of stainless steel. To increase the surface area of the device, it may be roughened by etching or other suitable means. Increasing the surface area implies that more material can be attached to the device, which may be desirable. It is though not necessary. Also, surface roughness or porosity implies that there will be coated surface areas on the device not exposed to friction towards the tissue if inserted by for example press fit. Thus, the surface of the implant device may be etched or have a rough surface. Polished surfaces also possess roughness on the nanometer to micrometer-scale, to the extent varying depending on the material and the material treatment procedures used. The surface topology of the implant device, particularly in terms of its roughness and porosity, may in itself affect tissue healing and implant success. Other surface treatment methods such as calcium mineral coating could also be used to increase surface roughness.

Preferably, the device is washed to remove organic and other contaminants, for example in hydrogen peroxide solution. For this cleaning step, a lot of cleaning methods are known in the literature, using various combinations of acids, bases, and organic solvents at different temperatures. The majority of silanisation procedures available in the literature recommend the use of a so-called ‘piranha’ cleaning for 1 h, immediately prior to the silanisation process. A piranha solution is a mixture of concentrated H₂O₂ (30% v/v) and concentrated H₂SO₄ (96% v/v), mixed in a ratio of respectively 1/4 or 3/7. As the piranha solution is a strongly oxidising solution, the hydrocarbon contamination will be removed from the surface of the substrate and thin oxide films will be further oxidised. Also ammonia or hydrochloric acid in combination with hydrogen peroxide solutions can be used. As an alternative to wet chemical cleaning, UV or UV-03 or RF-plasma based cleaning can also be applied. A majority of solid supports either carry hydroxyls on their surfaces or can be easily modified by chemical or electrochemical means to introduce such hydroxylic groups.

The first protein layer may be any suitable protein, such as fibrinogen. Preferably, a plurality of layers is used. For instance, a silane substance may be used to bind proteins onto the metal oxide (MOH<->) of the metallic device. Aminosilanes used in accordance with this invention are ones having the following general formula: H₂N—R′—Si(OR)₃ or H₂N—R′—SiX₃. In this formula, OR is a typical silane leaving group, such as methoxy, ethoxy, acetoxy and the like, as well as mixtures thereof, or the OR group can be a hydroxyl group and X can be a halogen such as chlorine and the like. The R′ group, which can be characterized as a spacer arm, is typically an alkyl group, an aromatic group, an ether, an ester or an imine-containing group. Exemplary R′ groups are the low to moderate length alkyl chains such as methyl, ethyl, propyl, butyl and up to as high as about C-9 or more. R′ aromatic groups include phenyl groups, and imine groups include aminopropyl groups and the like. Representative aminosilanes include 3-aminopropyltrimethoxysilane, 3-aminopropyltriethoxysilane (APTES), 2-aminoundecyltrimethoxysilane, aminophenyltrimethoxysilane, N-(2-aminoethyl-3-aminopropyl)trimethoxysilane, 3-mercaptopropyl triethoxysilane (MPTES), and trimethoxysilylpropyldiethylenetriamine.

For the deposition of silane molecules in a homogeneous manner, two methods can be applied, i.e. deposition from a solvent, also called liquid phase silanisation, and vapour phase deposition. Both methods are commonly used in silane technology. Most often, self-assembly is performed under solution conditions, at room temperature, using a wide variety of solvents. In the prior art methods vapour phase deposition of chlorosilanes may be preferred because it is believed to give rise to well-ordered monolayers, with a higher reproducibility and a higher stability, compared to liquid phase deposition.

In the production of the matrixes according to the invention, silane molecules may be deposited by means of liquid phase deposition. In that way, it is possible to form a well-defined thin layer. It has, however, to be understood that it may also be possible to deposit silane molecules using vapour phase deposition. In that case, however, the apparatus used to perform the vapour phase deposition should be such that it can be used at elevated temperatures and vacuum, as the silane molecules have a high boiling point and it is not easy to evaporate them.

For the silanisation of substrates comprising e.g. glass or metal oxide, the reaction times can be surprisingly short, i.e. between 10 minutes and 1 hour. The final stage of the formation of a well-ordered layer from alkylsilanes may take from 1 to 6 hours, or in some cases even longer, but is not necessarily required in the present application. A strong covalent binding may, by use of silane, be formed between the metal device and the first protein layer. More particularly, via covalent attachment of APTES to hydroxyl groups of the metal surface, a first fibrinogen layer may be immobilized onto the stainless steel surface. The functional groups on proteins which are available for covalent bonding are (1) amino (eta-amino groups of lysine and arginine and the N-terminal amino moieties of the polypeptide chains), (2) carboxyl groups of aspartic and glutamic acid and the C-terminal moieties, (3) phenol rings of tyrosine, (4) sulfhydryl groups of cysteine, (5) hydroxyls of serine, threonine and tyrosine, (6) the imidazole groups of histidine and (7) the indole groups of tryptophan. In practice, most of the covalent coupling reactions involve the amino, carboxy and mercapto moieties on the amino acids in the protein structure. It is also possible to replace APTES with some other substance that has an amino, carboxyl, SH or any other suitable chemical group. Amino groups of the APTES also react with aldehyde groups at one end of an aldehyde-based substance, such as glutardialdehyde. Glutaraldidehyde has a second aldehyde group that may be chemically bound to an amine terminal of the first protein layer. The silane attached to the device may also be one possessing a protein-binding functionality, such as an NHS-ester, conjugated carbonyls, epoxy, nitriloacetic acid, cyano, hydrazide, aziridine, sulfonylchloride, trifluoromethyldiaziridine, pyridylsulfide, N-acetyl-imidazole, vinylsulfone, arylazide, anhydride, diazoacetate, haloacetyl, benzophenone, isothiocyanate, isocyanate, halogen substituted benzene, pyridyldisulfide, biotin, protected carboxyl, protected amine, protected sulfohydryl, protected maleimide, allowing coupling of the protein directly thereto.

It would be possible to chemically bind a substance directly to the aldehyde groups of the glutaraldehyde, although depending on electron distribution and other physical properties of the drug substance, steric repulsions often makes it difficult to complete the monolayer. That is, less than the amount equaling a monolayer will result. It is often desirable to bind more drug substance than just one monolayer. Depending on the drug/substance to be used, the required drug-binding capacity of the matrix will vary. Optionally, only one, two, three etc protein layers may be desirable.

The first protein layer, or non-fibrinogen first layer (such as adhesive protein or carbohydrate) is thus attached to the surface by any of the above-mentioned means. The first protein may be fibrinogen, or it may be a different protein, for example a globular one such as albumin. Using a globular protein in the first layer may increase density and decrease ion leakage from the surface. This may be desired e.g. when apatites such as hydroxyapatite or tricalcium phosphate are used as substrates.

Further, carboxyl groups, such as but not limited to the free carboxyl terminals, of the first protein layer is activated by suitable substances, such as carbodiimide, e.g. ethyl-dimethyl-aminopropylcarbodiimide (EDC), and hydroxy-succinimide (NHS), to attract and by peptide bond formation capture more protein so as to form a second protein layer. For protein having carboxylate groups, activation is often achieved by contacting them with a solution of a carbodiimide coupling reagent and a succinimide reagent such as N-hydroxysuccinimide (NHS) or N-hydroxysulfosuccinimide (sNHS). The carboxylate groups are thus converted into NHS-ester or NHS-ester groups. Carbodiimide couplers include, for example, N-ethyl-N′-(3-dimethyl-aminopropyl)carbodiimide (EDC); dicyclohexylcarbodiimide (DCC); and diisopropylcarbodiimide (DIC).

The EDC activates the carboxyl groups, of the first protein layer, so that amino groups of the protein in solution may be chemically bound thereto. By repetition of the EDC/NHS activation procedure, a plurality of protein layers may be immobilized and cross-linked. The total thickness of the protein layer is increased by increasing the number of layers. For example, ten layers of fibrinogen may be about 280 Angstroms (Å) thick. 10 layers of fibrinogen may also be 500 Å thick. Differences depend on the exposition of the previous protein layer to the EDC solution, as well as the reactivity of the EDC itself (EDC efficiency decays, and has less than 2 hours of use after preparation/thawing), i.e. the efficiency in activating carboxyl groups. By cross-linking many layers of fibrinogen/protein, it is possible to thereafter embed an amount of drug substance greater or much greater than the amount equaling a monolayer. Typically, with 280 Å fibrinogen, 18 Å of bisphosphonate could be bound, and with 500 Å of fibrinogen, 45 Å bisphosphonate could be bound. It is to be understood that the drug(s) are not only disposed on top of the protein layer but are also mixed into the network of the layers, i.e. the matrix. The amount of Zolendronate or other drug with elevated calcium affinity can further be increased by alternating incubations of the surface with chemically bound Zoledronate in Ca-ion containing solution (typically CaCl₂) followed by incubation in a Zoledronate or other drug solution, and so forth. The incubation times are typically a few minutes to hours. The Zolenrdonate deposition is typically 5 Å (50 ng/cm²) per incubation cycle.

The amount of drug/substance incorporated into the matrix depends on a plurality of factors; properties of the drug/substance as well as properties of the protein/fibrinogen, and the conditions used, such as buffer, pH, time, concentrations, etc. In the examples typically a PBS-buffer is used, being standard in many laboratories, but an uncountable number of buffer-compositions would work equally well. Examples; Hepes, HBS, Tris, etc. paying attention to the properties of the drug/substance to be incorporated.

Common buffer compounds, as those in the following table, may be used.

| | | | Temp | | |
|---|---|---|---|---|---|
| | pKₐ | | Effect | | |
| Common | at | Buffer | (pH/ | Mol. | |
| Name | 25° C. | Range | ° C.)** | Weight | Full Compound Name |
| TAPS | 8.43 | 7.7-9.1 | −0.018 | 243.3 | 3-{[tris(hydroxymethyl)- |
| | | | | | methyl]amino}propanesulfonic acid |
| Bicine | 8.35 | 7.6-9.0 | −0.018 | 163.2 | N,N-bis(2-hydroxyethyl)glycine |
| Tris | 8.06 | 7.5-9.0 | −0.028 | 121.14 | tris(hydroxymethyl)methylamine |
| Tricine | 8.05 | 7.4-8.8 | −0.021 | 179.2 | N-tris(hydroxymethyl)methylglycine |
| HEPES | 7.48 | 6.8-8.2 | −0.014 | 238.3 | 4-2-hydroxyethyl-1-piperazine-ethane- |
| | | | | | sulfonic acid |
| TES | 7.40 | 6.8-8.2 | −0.020 | 229.20 | 2-{[tris(hydroxymethyl)methyl]amino}- |
| | | | | | ethanesulfonic acid |
| MOPS | 7.20 | 6.5-7.9 | −0.015 | 209.3 | 3-(N-morpholino)propanesulfonic acid |
| PIPES | 6.76 | 6.1-7.5 | −0.008 | 302.4 | piperazine-N,N′-bis(2-ethanesulfonic |
| | | | | | acid) |
| Cacodylate | 6.27 | 5.0-7.4 | | 138.0 | dimethyl arsenate |
| MES | 6.15 | 6.1-7.5 | −0.011 | 195.2 | 2-(N-morpholino)ethanesulfonic acid |
| Acetate | 4.76 | 3.8-5.8 | | 59.04 | — |

**Values are approximate

As indicated above, drug(s) or biologically active substance(s) may be immobilized to the protein layers. For example, aminated bisphosphonates, such as pamidronate, may be covalently bound to cross-linked protein layers. For instance, the amine group of a pamidronate molecule may be attached to the proteins of the matrix after activation of protein with EDC/NHS. Also suitable for covalent binding into the matrix are all kinds of peptide/protein-based drugs, such as enzymes, enzyme inhibitors, growth factors (and -inhibitors), affinity ligands, haptens, etc. Particularly, proteins such as bone morphogenic protein and fragments thereof and substances mimicking them are relevant when the present invention is used in bone.

In the below examples coating of devices have been accomplished through a plurality of incubations in different solutions under static conditions. The same matrix and drug incorporation can be achieved by use of flow or stirring during the reactions. Typically, this will shorten the incubation times required, preferably down to such times that the same EDC/NHS-solution can be used throughout all immobilisation steps (Within 1-2 hours). The experiments in the examples below where carried out at room temperature. Lowering the temperature will increase the life-time of the EDC-solution, as may be desirable. Increased temperature may be desired to increase yield of protein binding. Different temperatures during different steps of the matrix construction may therefore be suitable.

Implantation trauma often results in bone resorption that negatively affects the mechanical fixation of the implant device. One feature of bisphosphonates is that they inhibit bone resorption and likely decrease inflammatory activity of monocytes and macrophages, thereby improving over time the fixation of the implant device both when the drug is applied by local sudden treatment methods or by long-term systemic treatment. A localized surface dosage of about 120 ng/cm² is sufficient to increase fixation in the rat model used (Tengvall et al., Biomaterials 25, 2004). A rapid mechanical fixation of the implant device is important for the prognosis, not only for patients with compromised bone healing capacity but also for normal and healthy patients. A faster mechanical fixation likely improves the functionality of an implant due to an earlier and higher mechanical load uptake capacity. In parallel to this, it also decreases the thickness of the fibrous encapsulation and improves interfacial neo-vascularization. Early micromotion of the implant device is thus minimized, and the risk of late loosening reduced. Bisphosphonates released locally will stay within cm from the site of release for a long time (years). This contributes to augmented bone around the implant, giving long termed improved prognosis for the implants treated according to the invention (bisphosphonates)

Optionally, a second, third, and henceforth, drug substance is embedded into the matrix. This may be by any means mentioned above, and if the substance is a non-aminated such as ibandronate or zoledronic acid, typically by binding mechanisms such as EDC/imidazole coupling, hydrophobic and van der Waals interactions. The bonding may also involve magnesium, calcium ion (Ca⁺⁺), or other ions. Strontium (2⁺) may be used as a tool for electrostatic incorporation of a drug, and/or with the purpose of having Strontium itself delivered from the matrix. Thus, it is not required that the drug substance to be embedded possesses chemically reactive groups. Ibandronate was incubated with the matrix overnight (1 mg/ml, in PBS) resulting in a mass deposition equaling 42 ng/cm² (on the 280 Å fibrinogen+84 ng/cm² pamidronate matrix).

The fact that a non-covalently bound bisphosphonate layer is easily released from the implant device may have important advantages short time (up to 24 hours) after the implant device has been inserted. The insertion of implants into tissue often results in damage of the tissue matrix and disruption of the microcirculation in the immediate proximity of the implant. Matrix damage may, in case of bone, cause osteocyte apoptosis that may be implicated in osteoclast activation and remodelling, resulting in net bone resorption around the implant device. This likely leads to impairment of the implant fixation. The acute and rapid release of a second layer of bisphosphonate may specifically and effectively inhibit osteoclast activity and reduce bone resorption. This is of particular importance during the first couple of days or longer after insertion of the implant device. Further, there is a possibility that bisphosphonates have direct stimulatory effects on bone forming cells. As indicated above, bisphosphonates reduce the osteoclast precursor (monocytes/macrophages) activity in soft tissue, thereby lowering the non-acute, implant-prolonged, phase of the inflammatory process. Prolonged inflammatory activity is suspected to be one of the main reasons of fibrosis in soft tissue. Surface delivery of bisphosphonates shortens the inflammatory activity giving a faster wound healing process around implants. Non-amino bisphosphonate substances may be particularly useful to reduce the inflammatory reaction. This will improve implant functions, such as lowering of the voltage threshold in pacemaker-leads that contact heart muscles, and improve measurement of various soft tissue and body fluid properties, i.e. in biosensors.

The total thickness of bisphosphonate may be about 18 Angstroms or 45 Angstroms or more. The drug amounts mentioned are as measured by ellipsometry on flat surfaces. The microscopic surface of a typical implant device is typically 2, 3, 5, 10 or more times the macroscopic due to surface roughness and porosity. Considering the roughness of suitable implant surfaces, the amount of drug per macroscopic surface unit may be 2, 3, 5, 10 or more times the microscopic amounts given in this text. The drug release mechanism may rely on a spontaneous desorption of non-covalently bound drug and release of covalently bound ditto via hydrolytic and enzymatic cleavage. Approximately 30-50% of the drug amount may be desorbed during an overnight incubation in distilled water.

Regarding suture materials, they are made of e.g. polyamides such as nylon-6,6 and nylon-6, or poly(p-dioxanone) or polylactide/-glycolide. They are cleaned according to standard laboratory practice for 10 minutes by incubation in 70% ethanol followed by copious rinsing in distilled water and dried in nitrogen gas followed by 30 seconds exposure to UV. The structure surfaces become hydrolyzed during typically 3 hours in distilled water and treated one minute in a Radio Frequency Plasma chamber. Radio frequency plasma treatment roughens the surface of the suture material and generates charged and chemically reactive surface groups onto which for example spacers or proteins can be covalently attached. For example, surface carboxyl or amine groups may be formed on the suture via the surface activation procedures.

Thereafter, a linker molecule such as glutaraldehyde or ethyl-dimethyl-aminopropylcarbodiimide (EDC) is bound to the surface. One layer of protein/fibrinogen/non-fibrinogen substance from 1 mg/ml solution becomes covalently attached by the assistance of the linker molecule. More fibrinogen may subsequently be bound to this first layer in order to create a controllable but thin (thickness less than one micrometer) matrix into which the drug can be attached and/or associated.

The MMP-inhibitor, e.g. a tetracycline, is immobilized to the fibrinogen multilayer using the above-described EDC/NHS coupling technique. The suture specimens are stored in a solution of the same or a different MMP-inhibitor for up to 24 hours to allow additional loading of the matrix with loosely bound substance. The specimens are removed from the solution, blown dry in nitrogen, and kept sealed at ambient until used.

The thickness of the cross-linked fibrinogen layer is approximately 280 or 450 Angstroms and the MMP-inhibitor layers between 5 and 100 Angstroms. The MMP-inhibitor coated suture interferes with MMP at the surgical site, lowering the activity of the latter. The gradual release of the MMP-inhibitor provides a sustained effect resulting in maintained integrity of the otherwise degenerated tissue, for example collagen and tendon that surrounds the suture threads.

While the present invention is illustrated with preferred embodiments and their production, it is to be understood that certain substitutions and alterations may be made thereto without departing from the spirit and scope of the accompanying claims.

### Example 1

### Screws with Ten-Layers of Cross-Linked Fibrinogen and the Bisphosphonates Pamidronate and Ibandronate were Prepared, Followed by Deactivation with Gamma-Irradiation

Stainless steel screws, with threads measuring 1.7 mm in diameter and 3 mm in length were used. The screw specimens were cleaned for five minutes in acetone in an ultrasonic bath. The specimens were then etched during twenty minutes in 100% hydrofluoric acid (HF) and washed in a basic hydrogen peroxide solution at 80° C. for five minutes and finally rinsed in distilled water. Holes and asperities in the size range 0.1-100 micrometers were observed on the etched surface.

The screw specimens were put in a chamber with 0.2M 3-aminopropyltriethoxysilane H₂N(CH₂)₃Si(OC₂H₅)₃ (APTES from ABCR, Germany) and baked at 60° C. at 6 mbar for ten minutes. The temperature was then increased to 150° C. for one hour. The surfaces of the specimens were rinsed for two minutes in xylene (99% concentration, Merck, USA) in an ultrasonic bath. The surfaces were thereafter rinsed in xylene and stored in xylene no longer than one hour until the specimens were treated again. The so coated specimens were dried with flowing nitrogen and incubated for 30 minutes in freshly prepared 6% glutardialdehyde, OHC(CH₂)₃CHO, at room temperature in 0.2M Tris buffer, pH 9, to create a good environment for the reaction with aldehyde groups. The surfaces were then extensively rinsed and stored in the Tris buffer, pH 9.

Screws with ten layers of fibrinogen were prepared in the following way. The APTES and glutardialdehyde-coated specimens were incubated for thirty minutes in 1 mg/ml protein dissolved in phosphate buffered saline (PBS), pH 7.4. The specimen surfaces were thereafter extensively rinsed in PBS and incubated for thirty minutes in PBS at pH 5.5, containing 0.2M ethyl-dimethyl-aminopropylcarbodiimide (EDC, Sigma, USA). The specimen surfaces were again incubated for thirty minutes in a newly made 1-mg/ml protein solution in PBS, pH 5.5, thereafter rinsed in the PBS buffer and again incubated in the EDC/NHS solution. This procedure was repeated ten times to produce the ten-layer fibrinogen coating. Since the EDC/NHS solution is unstable at room conditions, new solutions were prepared every second hour.

Pamidronate disodium (AREDIA, 1 mg/ml in distilled water, Novartis, Sweden) was immobilized to the fibrinogen multiplayer using the above-described EDC/NHS coupling technique. An ibandronate solution (BONDRONATE, 50 mg/ml in distilled water, Roche, Switzerland) was adsorbed overnight on top of the pamidronate. The screw specimens were stored in the ibandronate solution for up to 24 hours until the specimens were inserted into rat tibia.

The thickness of the cross-linked fibrinogen layer was approximately 280 Angstroms and the pamidronate layers about 12 Angstroms. The more loosely attached ibandronate layer was about 6 Angstroms thick. The total amount of immobilized bisphosphonate was approximately 120 ng/cm² The amine groups of the pamidronate molecules were attached to the fibrinogen layers after activation of the fibrinogen film with EDC/NHS. Ibandronate adsorbed or attached to the immobilized pamidronate and about a monolayer (6 Angstroms) was formed during the overnight incubation.

The implant device was subjected to deactivation with gamma-irradiation at 25 kGy.

The pamidronate/ibandronate-coated surfaces of the stainless steel implant devices showed a mean of 28% (p=0.0009) increased pullout force at failure compared to non-bisphosphonate coated control specimens. The bone stiffness decreased by 8% compared to the control specimens although the change was not statistically significant. The pullout energy until failure increased by 90%, indicating drastically changed mechanical characteristics at the interface between the rat tibia and the bisphosphonate-coated specimen. This strongly indicates that the immobilized bisphosphonate layers of the implant device improved the metallic biomaterial fixation in bone.

### Example 2

### Screws with a Non-Clottable Fibrinogen Matrix of Ten-Layers of Fibrinogen and the Bisphosphonate Pamidronate are Prepared in the Following Way

Stainless steel screws, with threads measuring 1.7 mm in diameter and 3 mm in length are used. The screw specimens are cleaned for five minutes in acetone in an ultrasonic bath. The specimens are then etched during twenty minutes in 100% hydrofluoric acid (HF) and washed in a basic hydrogen peroxide solution at 80° C. for five minutes and finally rinsed in distilled water. Holes and asperities in the size range 0.1-100 micrometers could be observed on the etched surface.

The screw specimens are put in a chamber with 0.2M 3-aminopropyltriethoxy-silane H₂N(CH₂)₃Si(OC₂H₅)₃ (APTES from ABCR, Germany) and baked at 60° C. at 6 mbar for ten minutes. The temperature is then increased to 150° C. for one hour. The surfaces of the specimens are rinsed for two minutes in xylene (99% concentration, Merck, USA) in an ultrasonic bath. The surfaces are thereafter rinsed in xylene and stored in xylene no longer than one hour until the specimens are treated again. The so coated specimens are dried with flowing nitrogen and incubated for 30 minutes in freshly prepared 6% glutardialdehyde, OHC(CH₂)₃CHO, at room temperature in 0.2M Tris buffer, pH 9, to create a good environment for the reaction with aldehyde groups. The surfaces are then extensively rinsed and stored in the Tris buffer, pH 9.

Screws with ten layers of fibrinogen are prepared in the following way. The APTES and glutardialde hyde-coated specimens are incubated for thirty minutes in 1 mg/ml protein dissolved in phosphate buffered saline (PBS), pH 7.4. The specimen surfaces are thereafter extensively rinsed in PBS and incubated for thirty minutes in PBS at pH 5.5 (?), containing 0.2M ethyl-dimethyl-aminopropylcarbodiimide (EDC, Sigma, USA). The specimen surfaces are again incubated for thirty minutes in a newly made 1-mg/ml protein solution in PBS, pH 5.5, thereafter rinsed in the PBS buffer and again incubated in the EDC/NHS solution. This procedure is repeated ten times to produce the ten-layer fibrinogen coating. Since the EDC/NHS solution is unstable at room conditions, new solutions are prepared every second hour.

Pamidronate disodium (AREDIA, 1 mg/ml in distilled water, Novartis, Sweden) is immobilized to the fibrinogen multiplayer using the above-described EDC/NHS coupling technique.

The thickness of the cross-linked fibrinogen layer is approximately 280 Angstroms and the pamidronate layer about 12 Angstroms. The amount of immobilized bisphosphonate equals approximately 80 ng/cm². The amine groups of the pamidronate molecules are attached to the fibrinogen layers after activation of fibrinogen with EDC/NHS. Remaining EDC/NHS-activated.groups are inactivated by incubation with ethanol amine (C₂H₇NO), 1 mg/ml, pH 8.5, for 30 minutes. Thereafter, screws are incubated with NHS ester (Pierce EMCS (N-[ε-Maleimidocaproyloxy]succinimide ester), first dissolved in DMSO 4:1 (1 mg/0.25 ml), then diluted in PBS to final concentration of 1 mg/ml, pH 8, for 30 minutes. Screws are thereafter rinsed and dried in flowing nitrogen, and stored in sealed tubes. Tubes with screws are gamma-irradiated at 25 kGy without significantly reduced effect in extraction force from rat tibia at 14 days after incision surgery. Gamma-irradiation kills microorganisms, but it also changes chemical bindings within and in between the fibrinogen of the matrix, assuring non-clottable fibrinogen.

### Example 3

### Screws with a Non-Clottable Matrix of Eight-Layers of Fibrinogen and the Bisphosphonte Pamidronate are Prepared in the Following Way

Titanium screws were used coated with bisphosphonate. The screws were cleaned in acetone (100%, 3 min, at room temperature) and ultrasonicated 5 minutes and rinsed in distilled water. Screws were then further cleaned in UVO-chamber for 4 minutes times 4 (turned 90 degrees in between). Then they were incubated in 1% APTES in Xylene for 30 minutes, rinsed in Xylene and dried in flowing nitrogen. Thereafter screws are incubated in 6% glutaraldehyde in PBS pH 8.5 for 30 minutes, rinsed in destilled water and dried in flowing nitrogen. First layer of fibrinogen was attached by 30 minutes incubation in a 1 mg/ml fibrinogen solution in PBS, pH 7.4. Second and following layers were linked to the previous by use of repetive EDC/NHS and fibrinogen treatments, as described above. A total of 8 layers of fibrinogen result in an approximately 530 Å thick crosslinked fibrinogen matrix, equaling a surface mass of 6.4 microg/cm². The carboxyls in the matrix were further activated by EDC/NHS and thereafter incubated with Pamidronate (1 mg/ml, in H₂O). Remaining EDC/NHS-activated.groups are inactivated by incubation with ethanol amine (C₂H₇NO), 1 mg/ml, pH8.5, for 30 minutes. This was followed by incubation in C14-Alendronate (0.1 mg/ml, in PBS)+non labelled Alendronate (0.9 mg/ml in H₂O), to enable a concentration determination by beta-counter. Thereafter, screws are incubated with NHS ester (Pierce EMCS (N-[ε-Maleimidocaproyloxy]succinimide ester), first dissolved in DMSO 4:1 (1 mg/0.25 ml), then diluted in PBS to final concentration of 1 mg/ml, pH 8, for 30 minutes. The surfaces were finally rinsed in distilled water and dried in flowing nitrogen. The amount of immobilized Pamidronate was approximately 300 ng/cm². Both ellipsometry and radiolabelling techniques indicate the attachment, onto the Pamidronate coated fibrinogen matrix, of approximately 120 ng/cm² of Alendronate.

### Example 4

### Screws with a Non-Clottable Matrix of Two Layers of Fibrinogen and with the Bisphosphonates Pamidronate and Zoledonate were Prepared in the Following Way

The APTES and glutardialdehyde-coated specimens are incubated for thirty minutes in 1 mg/ml protein dissolved in phosphate buffered saline (PBS), pH 7.4. The specimen surfaces are thereafter extensively rinsed in PBS and incubated for thirty minutes in PBS at pH 5.5 containing 0.2M ethyl-dimethyl-aminopropylcarbodiimide (EDC, Sigma, USA). The specimen surfaces are again incubated for thirty minutes in a newly made 1-mg/ml protein solution in PBS, pH 5.5, thereafter rinsed in the PBS buffer and again incubated in the EDC/NHS solution. Pamidronate disodium (AREDIA, 1 mg/ml in distilled water, Novartis, Sweden) was immobilized to the EDC/NHS activated fibrinogen during an up to 120 minutes incubation. A second bisphosphonate, Zoledronate, was immobilized on fibrinogen amine groups by immersion of the fibrinogen matrix+pamidronate coated surfaces in 15 mg Zolendronate/ml+15 mg/ml EDC in 0.1M Imidazole, pH 6.0 during typically 1.5 hours-overnight incubations. Remaining EDC/NHS-activated.groups are inactivated by incubation with ethanol amine (C₂H₇NO), 1 mg/ml, pH 8.5, or Tris buffer pH 7, for 30 minutes. Thereafter, screws are incubated with NHS ester (Pierce EMCS(N-[ε-Maleimidocaproyloxy]-succinimide ester), first dissolved in DMSO 4:1 (1 mg/0.25 ml), then diluted in PBS to final concentration of 1 mg/ml, pH 8, for 30 minutes. The typical amount of pamidronate is 240 ng/cm² and Zolendronate 120 ng/cm².

### Example 5

### Screws with a Non-Clottable Matrix of Ten Layers of Fibrinogen and with the Bisphosphate Zoledronate were Prepared in the Following Way

The APTES and glutardialdehyde-coated specimens were incubated for thirty minutes in 1 mg/ml protein dissolved in phosphate buffered saline (PBS), pH 7.4. The specimen surfaces are thereafter extensively rinsed in PBS and incubated for thirty minutes in PBS at pH 5.5 containing 0.2M ethyl-dimethyl-aminopropylcarbodiimide (EDC, Sigma, USA). The specimen surfaces are again incubated for thirty minutes in a newly made 1-mg/ml protein solution in PBS, pH 5.5, thereafter rinsed in the PBS buffer and again incubated in the EDC/NHS solution. This was repeated until ten fibrinogen incubations were deposited. Zoledronate, was immobilized by immersion of the fibrinogen matrix coated surfaces in 15 mg Zolendronate/ml+15 mg/ml EDC in 0.1M Imidazole, pH 6.0 during typically 1.5 hours-overnight incubations. Remaining EDC/NHS-activated.groups are inactivated by incubation with ethanol amine (C₂H₇NO), 1 mg/ml, pH 8.5, or Tris buffer pH 7, for 30 minutes. Thereafter, screws are incubated with NHS ester (Pierce EMCS(N-[ε-Maleimidocaproyloxy]-succinimide ester), first dissolved in DMSO 4:1 (1 mg/0.25 ml), then diluted in PBS to final concentration of 1 mg/ml, pH 8, for 30 minutes. Typically, 240 ng/cm² of Zoledronate could be immobilized by this procedure.

### Example 6

### Sutures with a Non-Clottable Matrix of Ten Layers of Fibrinogen may be Prepared in the Following Way

Sutures prepared as above are incubated for thirty minutes in 1 mg/ml protein dissolved in phosphate buffered saline (PBS) at pH 7.4. The specimen surfaces are thereafter extensively rinsed in PBS and incubated for thirty minutes in PBS at pH 5.5, containing 0.2M ethyl-dimethyl-aminopropylcarbodiimide (EDC). The specimen surfaces are again incubated for thirty minutes in a newly made 1-mg/ml protein solution in PBS, pH 5.5, thereafter rinsed in the PBS buffer and again incubated in the EDC/NHS solution. This procedure is repeated ten times to produce the ten-layer fibrinogen coating but is not limited to this number of protein incubations. Since the EDC/NHS solution is unstable at room conditions, new solutions are prepared every second hour.

The MMP-inhibitor, e.g. a tetracycline, is immobilized to the fibrinogen multilayer using the above-described EDC/NHS coupling technique. Remaining EDC/NHS-activated.groups are inactivated by incubation with ethanol amine (C₂H₇NO), 1 mg/ml, pH 8.5, for 30 minutes. Thereafter, screws are incubated with NHS ester (Pierce EMCS(N-[ε-Maleimidocaproyloxy]succinimide ester), first dissolved in DMSO 4:1 (1 mg/0.25 ml), then diluted in PBS to final concentration of 1 mg/ml, pH 8, for 30 minutes. The suture specimens are stored in a solution of the same or a different MMP-inhibitor for up to 24 hours to allow additional loading of the matrix with loosely bound substance. The specimens are removed from the solution, blown dry in nitrogen, and kept sealed at ambient until used.

### Example 7

### Non-Clottable Fibrinogen was Prepared in the Following Way

Lyophilized fibrinogen (as provided by the supplier) was exposed to 5, 15, 25, or 35 kGy of γ-irradiation. The fibrinogen was then dissolved in PBS buffer, 1 mg/ml, and mixed with human thrombin solution from Sigma-Aldrich (USA) with enzymatic activity 0.5 U/ml, in PBS buffer and pH adjusted to 7.2.

The effect of gamma irradiation on fibrinogen clottability was analysed by three different methods, showing that the gamma-irradiated fibrinogen is non-clottable.

First method: By using the absorbance spectrometer Multiscan spectrum from Thermo Fisher Scientific (USA), the optical density at 280 nm was measured before and after coagulation. Samples of dissolved fibrinogen from each dose was transferred to PMMA cuvettes (Kartell, Italy) and measured three times, with PBS buffer as a blank. Then three samples for each gamma dose were mixed with thrombin to initiate coagulation and incubated for 30 min at room temperature. The formed gelatinous mass was then gently torn with a wooden stick, in order to get good separation of the mass and the supernatant after centrifuging at 4000 g for 15 min. The supernatant was removed and measured three times, alongside a PBS-thrombin solution as a blank. The quotient fibrinogen not participating in the network formation was compared between the different doses.

Except for 5 kGy, the results show an almost linear increase in free fibrinogen with higher irradiation dose. Fibrinogen irradiated with 25 kGy and 35 kGy show a significantly higher amount of free fibrinogen, 24% and 33%, respectively (p<0.05) compared to non-irradiated fibrinogen

Second method: Using the optical microscope Axio Observer D1 from Zeiss (USA), the network formation was examined in more detail. The formed network of fibrinogen irradiated with 0 kGy and 35 kGy was sampled onto microscopic slides for exposure. Captured images of the network were analysed with the Axio Vision software. The non-irradiated fibrinogen solution transformed to a jelly-like substance, and a dense, fine-masked, homogeneous network, was formed. Fibrinogen exposed to 35 kGy showed an inhomogeneous network, like a faint mist of light threads. The network is interrupted, forming only sporadic clusters of threads.

Third method: Visual inspection of samples was performed. The sample cups/cuvettes were tilted and held upside-down, in order to detect changes in consistency of the sample. The sample of non-irradiated fibrinogen transformed to a gelatinous clump and was unaffected of the upside-down treatment, i.e. the sample was clotted. Irradiated samples poured out when the cuvettes were held up-s ide down.

The method from Example 3 was used to prepare the fibrinogen matrix, rendering the same results with regard to matrix thickness (310 and 360 Å) and bisphosphonate incorporation (10 Å)

1-11. (canceled)

12. A non-clottable matrix on a solid support comprising immobilized and crosslinked fibrinogen.

13. The matrix on a solid support according to claim 12, further comprising in and/or on the matrix one or several biologically active compound(s) and/or pharmacological substance(s).

14. The matrix on a solid support according to claim 13, wherein the biologically active compound or pharmacological substance is non-aminated and bound by EDC/imidazole coupling, hydrophobic or van der Waals interactions.

15. The matrix on a solid support according to claim 14, wherein the non-aminated biologically active compound or pharmacological substance is ibandronate or zoledronic acid.

16. The non-clottable matrix on a solid support according to claim 12, wherein the matrix is composed of one or several fibrinogen layer(s).

17. The matrix on a solid support according to claim 16, wherein the several fibrinogen layers are selected from 2 to 100 layers.

18. The matrix on a solid support according to claim 12, wherein the fibrinogen has been rendered non-clottable stepwise during the production of the matrix.

19. The matrix on a solid support according to claim 12, wherein the fibrinogen has been rendered non-clottable prior to the production of the matrix.

20. The matrix on a solid support according to claim 12, wherein the fibrinogen of at least the outer layer(s) has been rendered non-clottable after the matrix has been constructed.

21. The matrix on a solid support according to claim 12, wherein the fibrinogen is selected from blood-derived fibrinogen and recombinant fibrinogen.

21. The matrix on a solid support according to claim 12, wherein the solid support is selected from the group consisting of orthopaedic devices, implants, stitches, stents, pins, screws, plates, and sutures.

22. The matrix on a solid support according to claim 12, wherein the fibrinogen matrix is attached to the solid support via a non-fibrinogen protein or substance.

23. The matrix on a solid support according to claim 13, wherein the one or several biologically active compound(s) and/or pharmacological substance(s) is (are) selected from the group consisting of tetracyclines, chemically modified tetracyclines, synthetic matrix metalloproteinase inhibitors, including those of the hydroxamate subgroup; cyclooxygenase inhibitors, including cyclooxygenase 2 specific inhibitors; nuclear factor kappa B inhibitors; lipooxygenase inhibitors; corticosteroids including glucocorticoids; macrolide antibiotics; hydroxymethylglutaryl coenzyme A reductase inhibitors (statins); angiotensin converting enzyme (ACE) inhibitors; angiotensin II receptor blockers (ARBs); bone morphogenic protein; aprotinin; gabexate mesilate; sulfasalazine; inhibitors of tumour necrosis factor alpha; and transforming growth factor beta inhibitors and bisphosphonates including compounds with the generic formula 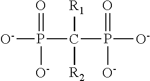
wherein R₁ and R₂ are independently selected from the group consisting of —H, —OH, —Cl, —CH₃, 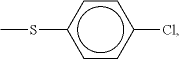
—CH₂—CH₂—NH₂, —(CH₂)₅—NH₂, —(CH₂)₂N(CH₃)₂, 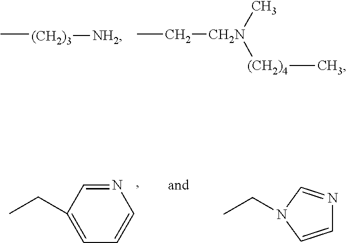
as well as pharmaceutically acceptable salts and hydrates thereof.
